(19) United States
(12) Patent Application Publication
(10) Pub. No.: US 2017/0202442 A1
(43) Pub. Date: Jul. 20, 2017

(54) MINIATURE OBSERVATION MIRROR IMAGING LENS STRUCTURE

(71) Applicant: DEX MEDICAL DEVICE CO., LTD., NEW TAIPEI CITY (TW)

(72) Inventor: CHIEN-LI CHANG, NEW TAIPEI CITY (TW)

(21) Appl. No.: 15/281,057

(22) Filed: Sep. 29, 2016

(30) Foreign Application Priority Data
Jan. 14, 2016 (TW) 105200540

(51) Int. Cl. A61B 1/05; A61B 1/267; A61B 1/045; A61M 16/04; A61B 1/00; A61B 1/06

(52) U.S. Cl. CPC A61B 1/05; A61B 1/00096; A61B 1/0684; A61B 1/045; A61M 16/0488; A61B 1/267; A61B 1/00045

(57) ABSTRACT

A miniature observation mirror imaging lens structure is disclosed, an outer tube body is in engagement with a front end of a bougie, and only an optical filter, lens, integrated circuit and circuit board are configured inside the outer tube body in sequence from front to rear, where a light emitting diode (LED) is configured on one side of the optical filter, electronic elements are soldered on the circuit board, a upper edge and lower edge thereof are respectively configured with a plurality of upper contacts and lower contacts; the integrated circuit can be linked with a control circuit on the rear end of the bougie after it is soldered on the upper contacts and conducting lines are soldered on the lower contacts, thereby allowing the diameter of the imaging lens on the front end of the bougie to be reduced for facilitating endotracheal intubation operation.

### TECHNICAL FIELD OF THE INVENTION

The present invention relates to a miniature observation mirror imaging lens structure, and more particularly to an endotracheal intubation visual bougie structure adapted to facilitate endotracheal intubation operation by reducing the outer diameter of an imaging lens on the front end of a bougie.

### DESCRIPTION OF THE PRIOR ART

General endotracheal intubation observation mirrors are functioned to allow the observation of a human organ state. A conventional observation mirror, as FIG. 1 shows, is configured with a soft and transparent endotracheal inner tube 1, in which a flexible bougie 2 is inserted, where the front end of the bougie 2 is configured with a tube body 21, inside which elements such as a optical filter, lens and optical element are configured, and on the rear end of which a connector 31 is configured, with the connector 31 being allowed to insert in a control circuit 3. Upon use, the endotracheal inner tube 1 together with the bougie 2 is inserted in a patient's mouth, and the control circuit 3 is connected to a host computer 4 with a display screen 41. Thereafter, the lens is adapted to capture the image of an organ after the optical element is light up, and image information is then transmitted to the connector 31 configured on the rear end of the bougie 2, with the connector 31 being inserted in the control circuit 3, thereby observing the image on the display screen 41 of the host computer 4. However, conventional observation mirrors are limited to the volumes of the elements, causing the imaging lens configured on the front end of the bougie 2 to be unable to be shrunk, the operation of medical staff is relatively inconvenient, and patients feel uncomfortable upon endotracheal intubation.

### SUMMARY OF THE INVENTION

The main object of the present invention is to provide a miniature observation mirror imaging lens structure, allowing the diameter of an imaging lens configured on the front end of a bougie to be reduced substantially for facilitating endotracheal intubation operation, and a patient's discomfort to be alleviated during endotracheal intubation operation.

To achieve the object mentioned above, the present invention proposes a miniature observation mirror imaging lens structure, an outer tube body being in engagement with a front end of a bougie, and only an optical filter, lens, integrated circuit and circuit board being configured inside the outer tube body in sequence from front to rear, wherein a light emitting diode (LED) is configured on one side of the optical filter, the lens below the optical filter, and the integrated circuit below the lens, allowing the LED and lens to be respectively connected to the circuit board; electronic elements are soldered on the circuit board, a upper edge and lower edge thereof are respectively configured with a plurality of upper contacts and a plurality of lower contacts, allowing the integrated circuit to be in contact with the upper contacts, and lower contacts to be in connect with conducting lines so as to be in connection with a control circuit configured on a rear end of the bougie, making a diameter of the imaging lens configured on the front end of the bougie reduced through the simplification of the elements configured on the front end thereof, thereby facilitating endotracheal intubation operation and alleviating a patent's discomfort upon the endotracheal intubation operation.

According to the structure mentioned above, an inner sleeve is configured between the optical filter and lens, thereby positioning the optical filter and lens.

According to the structure mentioned above, electronic elements are soldered on the circuit board, and a plurality of upper contacts and lower contacts are respectively configured on the upper and lower edges thereof, allowing the integrated circuit to be in contact with the upper contacts and conducting lines to be in connection with the lower contacts so as to be in connection with a control circuit configured on the rear end of the bougie.

### BRIEF DESCRIPTION OF THE DRAWINGS

FIG. 1 is a perspective view of a visual bougie of an observation mirror;

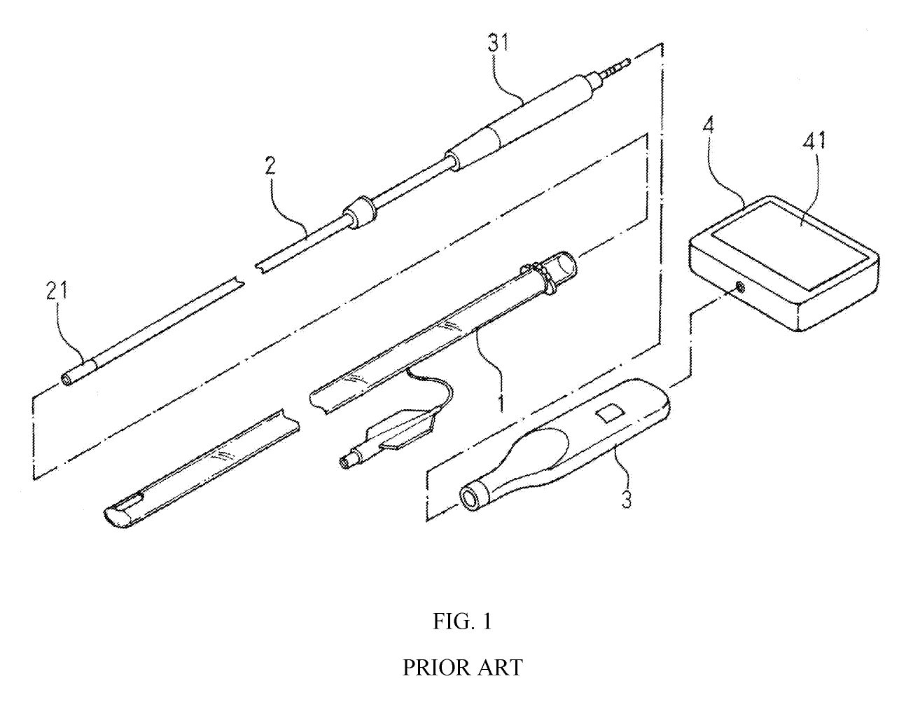

FIG. 2 is a perspective view of the present invention;

FIG. 3 is a cross-sectional view of the present invention;

FIG. 4 is a schematic view of a circuit board of the present invention after unfolded;

FIG. 5 is a perspective view of the present invention in a use state; and

FIG. 6 is a cross-sectional view of the present invention in an action state.

### DETAILED DESCRIPTION OF THE PREFERRED EMBODIMENTS

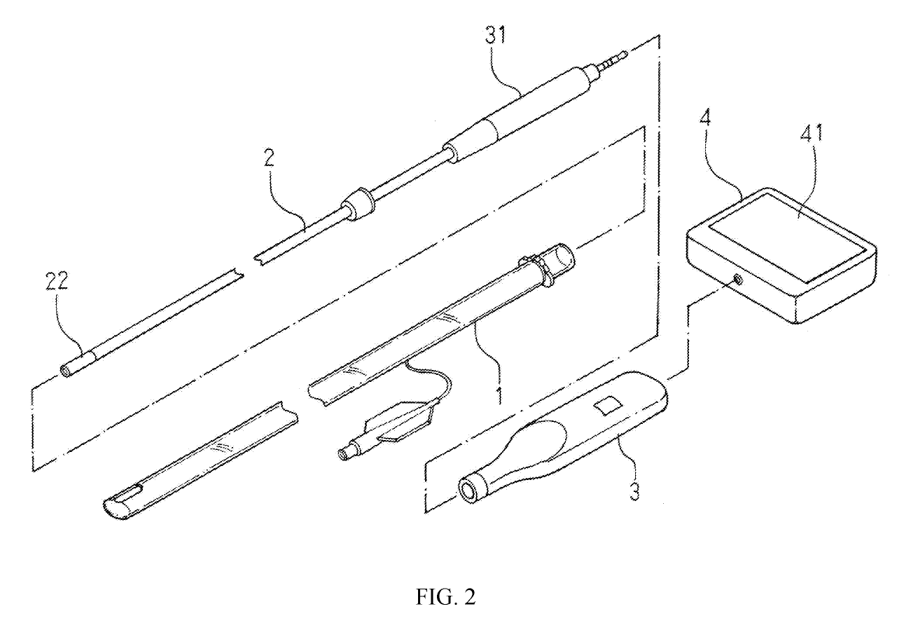

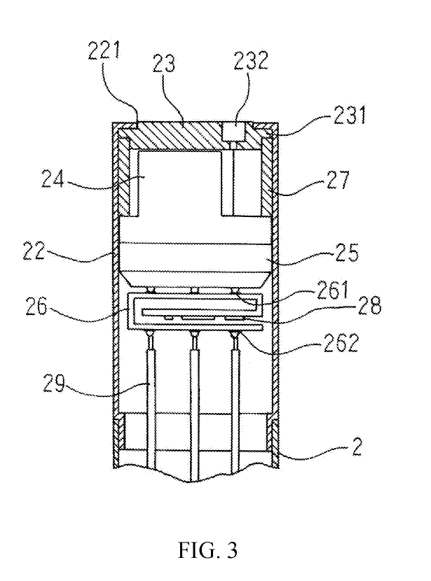

Referring to FIGS. 2 and 3, which respectively are a perspective view and cross-sectional view of the present invention, an observation mirror of the present invention, like conventional ones, is configured with a soft and transparent endotracheal inner tube 1, in which a flexible bougie 2 is inserted. Furthermore, an outer tube body 22 is engaged with the front end of the bougie 2, and a control circuit 3 is configured on the rear end thereof, where an optical filter 23, lens 24, integrated circuit 25 (Model OV6922 is adopted in the embodiment) and circuit board 26 are configured inside the outer tube body 22 in sequence from front to rear. In addition, an opening 221 is configured on one end of the outer tube body 22, and a wall portion 231 of a larger diameter on the optical filter 23, allowing the upper side of the wall portion 231 to be propped against the opening 221 of the outer tube body 22. Furthermore, a light emitting diode (LED) 232 is configured on one side of the optical filter 23, and the lens 24 below the optical filter 23, with an inner sleeve 27 being configured between the optical filter 23 and lens 24, thereby positioning the optical filter 23 and lens 24. Furthermore, the integrated circuit 25 is configured below the lens 24, allowing the LED 232 and lens 24 mentioned above to be respectively connected to the circuit board 26.

The circuit board 26 allows electronic elements 28 to be soldered thereon, the upper edge and lower edge of which is respectively configured with a plurality of upper contacts 261 and a plurality of lower contacts 262, allowing the integrated circuit 25 to be in contact with the plurality of upper contacts 261 and the plurality of lower contacts 262 to be in connection with conducting lines 29 so as to be in connection with the control circuit 3 configured on the rear end of the bougie 2, thereby allowing the outer diameter of the front end of the bougie 2 to be reduced for facilitating endotracheal intubation operation. In addition, in the embodiment, the circuit board 26 is circuitously, continuously folded, making the upper contacts 261 and lower contacts 262 positioned respectively on the upper and lower edges.

The miniature observation mirror imaging lens structure is thus formed through the combination of the components mentioned above. Upon use, the bougie 2 is inserted into the endotracheal inner tube 1, the endotracheal inner tube 1 is then inserted into a patent's mouth together with the bougie 2, and the control circuit 3 is connected to a host computer 4 with a display screen 41. Thereafter, the lens 24 captures the images of a organ through the illumination of the LED 232, and image information is then transmitted to the control circuit 3 configured on the rear end of the bougie 2, thereby observing the images on the display screen 41 of the host computer 4. Therefore, the diameter of the imaging lens configured on the front end of the bougie 2 can be reduced substantially for facilitating the endotracheal intubation operation and reducing the patent's discomfort during the intubation. In the embodiment, the area of the circuit board 26 is equal to or smaller than the one of the integrated circuit 25, a large portion of elements are moved rearward, and the area of the lens 24 is further equal to or smaller than the integrated circuit 25, allowing the diameter of the imaging lens configured on the front end of the bougie 2 to be reduced substantially.

The diameter of the circuit board 26 mentioned above may be limited below 2.9 mm, and further, the outer tube body 22 below 3.5 mm.

In the embodiment, the integrated circuit 25 mentioned above is Model OV6922.

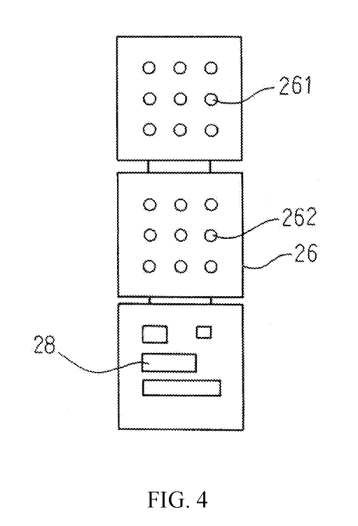

Referring to FIG. 4, which is a schematic view of the unfolded circuit board of the present invention, the circuit board 26 of the present invention, in the embodiment, includes a printed circuit part for the soldering of electronic elements 28, and the upper contacts 261 and lower contacts 262 in connection with the printed circuit part, allowing the printed circuit part and the upper contacts 261 as well as lower contacts 262 to be circuitously, continuously folded, thereby making the upper contacts 261 and lower contact s 262 positioned respectively on the upper and lower edges thereof.

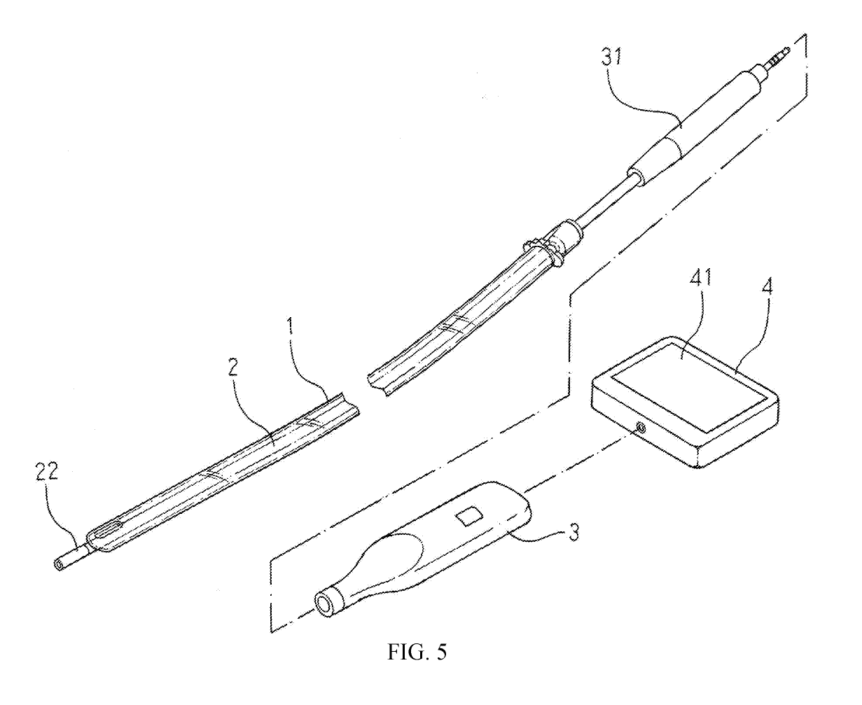

referring to FIG. 5, which is a perspective view of the present invention in a use state, the bougie 2, upon use, is inserted into the endotracheal inner tube 1, and the endotracheal inner tube 1 is then inserted into a patient's mouth together with the bougie 2 since the endotracheal inner tube 1 is soft and the bougie 2 is flexible. Therefore, the endotracheal inner tube 1 can be bended with a human organ structure together with the bougie 2 for facilitating the in-depth thereof while being inserted in the patient's mouth, thereby capturing images through the front end of the bougie 2.

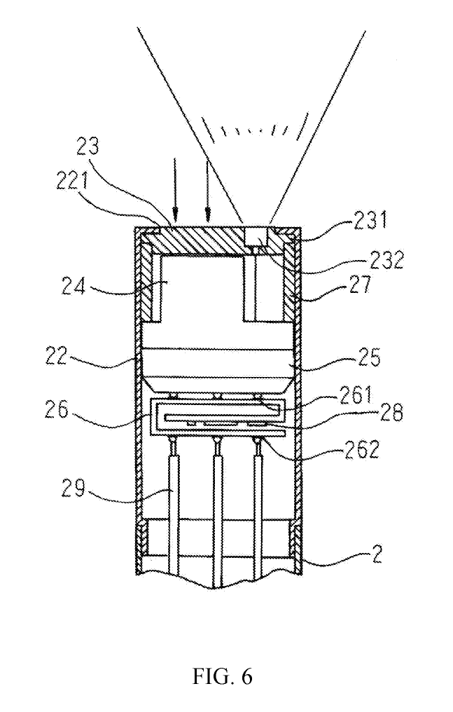

Referring to FIG. 6, which is a schematically cross-sectional view of the present invention in an action state, and FIG. 2 again, the LED 232 configured on the front end of the bougie 2, upon use, is lighted up through the control circuit 3 to illuminate an organ, allowing the lens to capture the images thereof clearly, and image information is then transmitted to the control circuit 3 on the rear end of the endotracheal inner tube 1 through the conducting lines 29 for the processing of the control circuit 3, thereby observing the images on the display screen 41 of the host computer 4.

To sum up, the present invention uses the outer tube body in engagement with the front end of the bougie to coordinate with the optical filter, lens, integrated circuit and circuit board configured inside the outer tube body to form a miniature observation mirror imaging lens structure, allowing the outer diameter of the front end of the bougie to be reduced substantially and facilitating the alleviation of a patient's discomfort upon endotracheal intubation operation.

I claim:

1. A miniature observation mirror imaging lens structure, an outer tube body being in engagement with a front end of a bougie, and only an optical filter, lens, integrated circuit and circuit board being configured inside said outer tube body in sequence from front to rear, wherein a light emitting diode (LED) is configured on one side of said optical filter, said lens below said optical filter, and said integrated circuit below said lens, allowing said LED and lens to be respectively connected to said circuit board; electronic elements are soldered on said circuit board, a upper edge and lower edge thereof are respectively configured with a plurality of upper contacts and a plurality of lower contacts, allowing said integrated circuit to be in contact with said upper contacts, and lower contacts to be in connect with conducting lines so as to be in connection with a control circuit configured on a rear end of said bougie, allowing a diameter of said imaging lens configured on said front end of said bougie to be reduced through the simplification of said elements configured on said front end thereof, thereby facilitating endotracheal intubation operation and alleviating a patent's discomfort upon said endotracheal intubation operation.

2. The structure according to claim 1, wherein an opening is configured on one end of said outer tube body, said optical filter haves a wall portion of a larger diameter, allowing a upper side of said wall portion to be propped against said opening of said outer tube body.

3. The structure according to claim 1, wherein an inner sleeve is configured between said optical filter and lens so as to position said optical filter and lens.

4. The structure according to claim 1, wherein said circuit board is circuitously, continuously folded, allowing said upper contacts and lower contacts to be respectively positioned on upper and lower edges thereof.

5. The structure according to claim 1, wherein the area of said circuit board is equal to or smaller than the one of said integrated circuit, further allowing the area of said lens to be equal to or smaller than the one of said integrated circuit, thereby making the outer diameter of a front end of said imaging lens reduced substantially.

6. The structure according to claim 1, wherein the outer diameter of said circuit board is below 2.9 mm.

7. The structure according to claim 5, wherein the outer diameter of said circuit board is below 2.9 mm.

8. The structure according to claim 1, wherein the diameter of said outer tube body is below 3.5 mm.

9. The structure according to claim 1, wherein said integrated circuit is an integrated circuit of Model OV6922.
